(19) United States
(12) Patent Application Publication
(10) Pub. No.: US 2010/0267841 A1
(43) Pub. Date: Oct. 21, 2010

(54) Composition for Topical Use Containing an Extract of Stryphnodendron; Its Preparation As Well As Its Application

(75) Inventors: Suzelei de Castro Franca, Ribeirao Preto/SP (BR); Joao Carlos Nuncs de Oliveira, Ribeirao Preto/SP (BR); Luiz Pasqualin, Ribeirao Preto/SP (BR); Lucelio Bernades Couto, Ribeirao Preto/SP (BR); Raphael Carlos Comelli Lia, Araraquara/SP (BR)

Correspondence Address: SMITH HOPEN, PA, 180 PINE AVENUE NORTH, OLDSMAR, FL, 34677, US

(73) Assignees: AERP ASSOCIACAO DE ENSINO DE RIBEIRAO PRETO, Ribeirao Preto/SP (BR); APSEN FARMACEUTICA S.A, Ribeirao Preto/SP (BR)

(21) Appl. No.: 12/823,397

(22) Filed: Jun. 25, 2010

Related U.S. Application Data
(62) Division of application No. 11/381,655, filed on May 4, 2006.

(51) Int. Cl. A61K 31/05; A61P 17/02

(52) U.S. Cl. 514/731

(57) ABSTRACT

The present invention relates to topical compositions containing extracts of the plant genus Stryphnodendron as well as their preparation and application. The compositions contain between 1% and 6% of total phenols and are useful for the treatment of cutaneous wounds.

### CROSS REFERENCE TO RELATED APPLICATIONS

This application is a divisional of prior application Ser. No. 11/381,655, entitled “Composition for Topical Use Containing an Extract of Stryphnodendron; Its Preparation As Well As Its Application”, filed on May 4, 2006, the contents of which are incorporated herein by reference.

### FIELD OF THE INVENTION

The present invention belongs to the field of pharmaceutical segment, particularly consisting of a phytotherapeutic pharmaceutical formula for the treatment of ulcers, either those that obstruct or decrease blood irrigation, and more particularly pressure ulcers and varicose ulcers (both venous or arterial), besides the preparation process and its application, being said phytotherapeutic pharmaceutical forms featured for the reason of having gross extract and portions of plants of the gender Stryphnodendron.

### BACKGROUND OF THE INVENTION

As it is of general knowledge, for thousands of years wounds have been treated through aiming at enhancing the natural scar healing process. It is known that some wounds will become scars healed by themselves whilst others will resist from any efforts to do so. These wounds become cases that will lead persons to diabetes, cancer, varicose ulcers and pressure ulcers. Ulcers that originate from a long period of time lying in bed are best known as crusts.

In accordance with Modolin and Bevilacqua (1985) scar healing is a physiological process that begins through an inflammatory response featured by an increase on the blood flow, capillary permeability and migration of leukocytes to the harmed region. The capillary permeability provides the extravasation of plasma and its components thus forming the inflammatory waste material.

At the start a wound is filled by clots, insoluble whitish proteins and waste material that form up a crust that isolates the wound from the external side almost at once (COTRAN, 1989). The neutrophils and macrophages are the first cells to migrate into the wounded region in a response from the organism against the bacterial invasion (RUNNELS et. al., 1976) and when the cells destroy themselves and waste away the bacteria degenerate themselves forming up the pus with the dead remaining tissue (GUYTON, 1991).

When the granulation tissue contracts itself it retracts the edges of the wound in the skin towards the central part of the wound thus allowing the area to be reformed and become smaller. When granulation is excessive a delay occurs to the scar healing. Prevention can be obtained by encouraging a subcrustal scar healing when the formation of a crust wound encourages the scar healing process. The crust appearance can occur by the use of several substances such as tannins. When the wound waste material lingers, desegregation on the crust occurs, thus allowing the development of germs between the wound and the granulation tissue (OLIVEIRA 1992).

As it is of general knowledge, pressure ulcers or crusts are excavated lesions that might reach variable depths (named “grades”) reaching the skin and as well even the muscles and bones. These crusts are caused by a closure in the blood circulation as a consequence of prolonged pressure on the bones of the musculature and on the neighboring region. The obstruction blocking the blood circulation results from lack of normal movements of the patient as well as a deficit of sensitivity. These are the basic reasons of the necrosis areas that further develop into the appearance of wounds.

The crusts can take place in several parts of the body. In most cases the salient bones force up the soft tissues of the patient against hard surfaces that encourage the lesion to appear. For instance, a patient who is lying in bed for a long time exercises pressure on his/her heel through the weight of his/her leg thus forcing up a small area of the skin. This pressure is sufficient to block the blood vessels between the bone of the heel and the skin of the surface thus forming up a crust ulcer on the heel. In his/her turn, the patient who stays for a long time lying on his/her back without ever moving himself/herself will exercise pressure on the sacral bone thus allowing the crust to originate from there. The sacral bone is where most crusts are found. Other regions such as the clavicles, elbows, ribs and occipital bones are also very subject to the appearance of crusts. Therefore, the appearance of crusts begins with the prolonged pressure on a determined part of the body up to the time when the wound is originated by a lesion of surrounding skin.

In their turn, the varicose ulcers (venous or arterial ones) are a consequence from a backlog of venous blood in the skin. This backlog determines their thickness, the reduction of subcutaneous fat lard and brown stains. In the arterial hypertension, diabetes and arterial thrombosis the obstruction of the skin arteries or of the profound tissues occurs, thus originating the wound. Infectious ulcers are painful, show inflammatory characteristics and have pus secretion.

The ulcers (pressure or varicose ones) may be described in accordance with the characteristic stages of tissue loss usually classified into grades in accordance with the standards of the Quick Reference Guide for Clinicians. These grades include:
Grade I—skins shows hyperemia (redness)
Grade II—the wound penetrates the skin (epidermis and/or “dermis) causing also damage or necrosis in the subcutaneous tissue. The lesion clinically appears as a superficial surface ulcer with or without damage to the adjacent tissue.
Grade III—loss of skin and tissues that extends to the subcutaneous tissues; and
Grade IV—the lesion reaches the muscle articular and bone tissues.

The damaged tissue with waste material is a fiber optic tissue mostly composed of non-organized collagen fibers. This type of tissue has little vascularity, little nervature and little elasticity. Because of this absence of nervature and loss of elasticity, this type of tissue is susceptible to a series of injuries and resists scar formation due to the lack of vascularity.

Numerous types of treatment are used for pressure ulcers that involve preventative measures. These treatments include: a) local treatment or care using special beds, water mattress, etc; b) patient treatment that includes asepsis, nutrition improvement, care and fight against local infections, etc; and c) use of physical therapies over the affected area with electric stimulation, ultrasound, laser therapy and others.

In a general manner the scar healing process that naturally takes place in response to the injury includes the inflammatory response, angiogenesis, synthesis and deposition of collagen and formation of the scar. Although tissue repair and regeneration occur without an intervention, scar healing might occur due to the acceleration of the collagen synthesis. Thus, the scar healing of wounds involves a series of complex biochemical and cellular events which finally promote a retraction, closing and scar healing of the lesion.

The term “revitalization” is used as a means to restore the vascularity and elasticity of the tissue that had been injured and crusted. The term “injury” is used as a method of provoking the formation of a wound caused by surgery, traumas, ulcers, burns, chemical agents and virus or bacterial infections. The term “crusted” tissue is a type of fiber optical tissue or collagen formed during the scar healing of the wound or other pathological processes. The crusted scarred tissue is a fiber optical tissue produced by hundreds of non-organized collagen fibers and is formed by injuries or inflammation in the tissue site.

The contamination of a wound may be a result from the direct contact with the infected object or by incoming dirt, dust or exogenous microorganisms from the patient's skin or gastro-intestinal area. For instance, it has been recognized that despite adopting effective measures to avoid infections, practically all burns start up a colony of bacteria within a period of 12-24 hours.

In a general manner, infection bars the scar healing of the wound due to the provocation of tissue damage and inflammation. Subsequently, the recovery of the wound is damaged by a progressive inflammation, formation of waste materials, release and activation of enzymes, generation of free radicals, consumption of oxygen and loss of nervure. Therefore, cares to prevent inflammation might lead to the scar healing of the wound while not risking the ability of the tissue to resist the infection or inhibit the essential function of the macrophages.

The prior art teaches that the medical protocols utilized for scar healing of wounds are varied in their benefits and limitations. The actives available for topical therapeutics include the use of: antibiotics; bismuth salts; carbohydrates; hormones; plasma; zinc and tannic acid; treatments with electric stimulation; hyperbaric oxygenation; ultrasonic therapy and laser.

Still taking into consideration the traditional protocols the wounds treatment has been intensified in relation to the use of natural products to aid scar healing, such as copaiba oil (Coorea 1984; Eurides & Mazzanti, 1995); papaina (Sanchez Neto et al., 1993); sugar (Prata et al., 1988); collagen (Abramo, 1990) and vitamin A (Bondi, 1989).

The Barbatiman (Stryphnodendron polyphyllum and S. adstringens; Martius), particularly the phytotherapuetic ones dealt with in this development of innovated pharmaceutical formulation, comprise a leguminous mimosoidea arboreal from the Brazilian flora that is found from the State of Para up to the State of Sao Paulo. The barks are thick and have a binding effect that is related to the presence of active principles like tannins. Extracts from the barks of these species can be used in the preparation of scar healing phytotherapeutics due to the similarity of the chemical composition.

It was mentioned by the Brazilian Pharmacology (1959) that the barks from the plant have tannins that hold important pharmacological activities on mice like anti-inflammatory actions (LIMA, 1998) and scar healing in dermis wound (PANIZZA, 1998 and EURIDES, 1996), in gastric ulcer (FAVARETTO et al., 1985) and duodenal (RIZZINI & MORS, 1976). Tannins also assist in the treatment of eczema with the advantage of not generating collateral effects usually observed in therapies using high dosages of glycocorticoids (MROWIETS et al., 1991).

In accordance with Mello (1998) the main features of the extracts made from the barks of Barbatiman is found in the richness of tannins. In '1996 Mello et al. (MELLO, 1998) isolated and identified 22 compounds on gross extract of barks from Barbatiman. All were condensed type tannins that make up 25-30% of the plant bark and can be used in several biological activities. (PANIZZA et al., 1998). Proanthocyanidans, prorobinetinidins, flavan-3-ols and prodelfinidins were the chemical portions studied. According to Lima et al. (1998) the anti-inflammatory activity of Barbatiman is attributed to the presence of proanthocyanidins.

Other studies have investigated the composition of tannins in three species of Barbatiman, that is, the S. polyphyllum, the S. adstringens and the Dimorphandra mollis (SANTOS, 2002) wherein the same were already evaluated chemically. It is to be emphasized that the D. Mollis is known as Barbatiman, nevertheless it differs chemically from the two aforesaid specimens as shown below:

Chromatographic Analysis of Barbatiman Extract after Hydrolysis

| Species | Plant | Delphinidina | Cyanidina | Galic Acid | Flavonols |
|---|---|---|---|---|---|
| S. adstringens | Bark | + | − | + | − |
| | Leaf | + | − | + | + |
| S. polyphyllum | Bark | + | − | + | − |
| | Leaf | + | − | + | + |
| D. mollis | Bark | − | + | − | + |
| | Leaf | − | − | − | + |

In accordance with Haslam (1996) tannins combine themselves with the proteins through chemically well defined links and chains which provide the binding feature. This complex tannin/protein and/or polysaccharide forms a protective coating on the skin or on the mucosa damaged. Under this coating the natural process of recovering the wounds, burns and inflammations might occur.

Tannins also hold inhibiting effects on bacteria and fungi (antimicrobial activity), based on the assumptions that the tannins inhibit the enzymes of the bacteria and fungi through acting with the substrates of these enzymes. Furthermore, tannins act on the cellular membranes of microorganisms through changing their metabolism and forming complexes with metallic ions thus decreasing the availability of essential ions for the metabolism of the microorganisms (SIMOES, 2000).

Neto et al. (1996) compare the scar heating action of Calendula officinalis L and Stryphnodendron barbatiman (Vellozo) Martius (Barbatiman) in the treatment of varicose ulcers and skin lesions in humans. The results show that treatments with calendula and as well with barbatiman are efficient in the recovery of burn lesions and in varicose ulcers once the association allowed a faster scar healing.

Earlier studies disclose that one of the physiotherapeutic alternatives for accelerating the tissue process is the use of laser. The laser is a special form of electromagnetic energy that operates in the region of visible electromagnetic spectrum or infrared. There are many different types and powers of lasers.

In accordance with Baxter et al. (1994) the success with the use of laser is due to the responses induced in the tissues such as the reduction of edema, a decrease in the inflammatory process, an increase in phagocytosis, and an increase in collagen synthesis. The success is most likely due to the biological effects related to the proliferation of cells involved in scar healing, mainly fibroblasts and macrophages. (O'KANE et al., 1994).

In vitro studies are reported more frequently in the reduction of scar healing time of wounds in the cutaneous and mucosal extracts particularly on the proliferation and activation of the protein synthesis of fiber blasts, the principal cell responsible for the tissue restoration (ABERGEL, 1984; BOULTON, 1986; HALLMAN, 1988; LOEVSCALL & ARENHOLT-BINDSLEV, 1994).

Laser is a non-invasive instrument important in the recovery of ulcers (SCHINDL et al., 1999) and of numerous etiologies, including venous and diabetes ulcers (ALGAN et al. 2001). The scar healing time is dependent on the size and cause of the ulcers.

In 1991 Arantes disclosed that the application of low intensity laser in dermis lesions of lower limbs induced cures without reoccurrence in 70% of patients with 30% having improved diagnostically when compared to normal clinical proceedings. The normal clinical proceedings disclosed a cure rate of 38%; an improvement rate of 48%; and absence of recovery rate of 14%.

Besides the treatment with topical products for scar healing, physiotherapeutic apparatuses such as ultrasound (US) and low intensity laser can serve as alternatives to the treatment in tissue recovery.

The ultrasound comprises another method widely used to accelerate tissue recovery process, being its functioning due to thermal and non-thermal effects (mechanical) generated by the ultrasonic waves on the damaged tissues (STARCKEY, 2001; DYSON, 1992). The therapeutic effects carried out by the low intensity US are usually due to the non-thermal effects produced by stable waves, acoustic currents, micro currents and cavities (DYSON, 1987).

The skin, in addition to its functions as a barrier, is also a target for drug administration with the advantage of decreasing possible adverse effects caused by oral means of administration or other invasive means of administration (MATCHED, 2002). Nowadays there is an increasing interest in ultrasound since it allows the transdermal transport of drugs with low molecular weight or even of proteins with a high molecular weight such as insulin. This transdermal delivery would allow injection of insulin to be avoided thus preventing the occurrence of aches and possible permanent damages to the skin.

Recent studies have been carried out with the purpose of altering the cutaneous permeability, including the use of chemical “facilities” (MORGANTI et al., 2001) and the physical “facilities” like iontophoresis, an electrotherapeutical technique that utilizes the galvanic current to facilitate the intercutaneous penetration of therapeutic substances (BARRY, 2001) and, of a widespread use, phonophoresis or sonophoresis (MATCHED & BOUCAUD, 2002).

This technique is comprised of the association of ultrasound with several drugs used as a means of coupling in the form of gel or ointments thus changing its diffusion across the corneous extract. The US increases the coefficient of diffusion of the corneous extract to hydrophobic molecules of low and high molecular weight (MITRAGOTRI, 2001; JOSH1 & RAJE, 2002). Such corneous extract can be applied together with chemical facilities thus (TEZEL et al., 2002) enhancing the absorption of several medicines. In this aspect Skanem & Fentner (1984) disclosed that the ultrasound was employed in different previous works for offering localized conditions in the skin which favored the diffusion of drugs.

Thus, the ultrasonic heating, observed in an insignificant magnitude is responsible for a modest alteration in the intensity, frequency and modes of drugs transportation. The grade of generated heat is considerably influenced by subtle factors like the movement of the transductor, the anatomical site of application, the quantity and type of vehicle or means of coupling pressures and radiations, the forces generated by the micro currents and the formation of steam or gas bubbles in liquid.

Even before the previous studies were described in the literature, it was noticed that the diffuse and popular use of Barbatiman, with or without association of the Calendula plant, was still of empirical procedure. For its application into a site, an infusion of Barbatiman's bark was made, and applied directly to the wound thus resulting in a methodology without any kind of penetration control, local moisture control, or control of the contents of contamination by added agents. There were no pharmaceutical forms that were enabled to measure dosages or control clinical methodologies.

This type of procedure could not be repeated in those patients who hold rebel wounds such as ulcers, including varicose and pressure ulcers. Asepsis and the prevention against inflammations and infections are the most important topics during the scar healing treatment of wounds. It is necessary for the said wounds to produce tissue recovery and regeneration. Scar healing occurs due to the acceleration of collagen synthesis.

The present invention aims to allow the use of Stryphnodendron barks' properties (Barbatiman) in a pharmacological way in both human and veterinary application. The present invention relates to a phytotherapeutical pharmaceutical form for scar healing, as well as its process of preparation, application, active and derived composition. It was demonstrated that formulating a product containing extracts and portions of a medicinal plant, particularly Stryphnodendron polyphyllum or Barbatiman which are native to Brazil and have a high content of total phenols and tannin constituents, would be beneficial. A high content of total phenols and tannin constituents developing in portions of dye and powder have ideal physical and chemical features that enable the formulation of products that can receive the ingredients and can be used as vehicles that are capable of being dosed clinically as well as pharmaceutically.

The great advantage of using formulations that contain extracts of Stryphnodendron is related to the scar healing power which is enhanced by the antimicrobial action which keeps lesioned sites with a minimized level of microorganism thus favoring the scar healing.

The described process was carried out from dyes that were extracted from the Stryphnodendron's barks, concentrated on rotovaporator and dried by forced circulation of air. The barks were subject to quantitative analysis for determination of the contents which were found to be predominantly tannin and total phenols (AOAC, F. Bras. IV supplement 35, pages 30-2). The average amount of total phenols varies between 40-50%. Following, the preparation of pharmaceutical forms was carried out with a quality control that obeyed the specifications described in the 4th Edition of Brazilian Pharmacopy.

The pharmaceutical form was made of different concentrations of actives. Through pre-clinical studies the highest efficiency concentration was verified in the process of scar healing. One hundred fifty lesions were evaluated from which 60% had scarred completely.

Literature has disclosed that the scar healing process is affected by the presence of microorganisms, most of them multiresistant.

In vitro microbiological assays using standard specimens and field species isolated from infected crusts has shown that the Stryphnodendron's extracts hold GIM 62.5 g/mL for gram positive bacteria and 250 g/mL for gram negative bacteria. The tested pharmaceutical forms contain concentrations of active substances 400 and 100 times to GIM of positive and negative gram respectively.

Clinically it was observed that the infected crusts also responded to the treatment with the active and scar healed without the need of complementary use of antibiotics.

During the clinical study of the Barbatiman's physiotherapeutic pharmaceutical formulation, the patients under treatment were subject to macroscopic and microscopic examinations of organs (stomach, kidneys, liver and spleen); laboratory examinations like hemograms; and biochemical assays of renal and hepatic function before, during and after treatment. The results proved that the phytotherapeutical form did not affect the biochemical and hematological parameters analyzed as shown in FIGS. 1-8.

The results of therapeutical efficiency that were obtained acknowledge the success of the form of the phytoderived medicine that is constituted of 3% of Stryphnodendron's extracts, in the way that it will be defined further on. The medicine was used in the Clinical Study of Scar Healing Action's Evaluation of 150 crusts with different grades of tissue damage:

Treated lesions were predominantly of Grade I (61.2%) and Grade II (20.1%). The medicine was successful in the complete scar healing of 58.8% for the lesions of Grade 1 and 35.6% for the lesions of Grade II. The evolution in the scar healing process showed that the tested phytotherapeutical form provided the scar healing of over 70% of treated lesions within a maximum period of 2 months. The time for scar healing varied in accordance to the grade and site of lesion. For example, the crust of a Grade I lesion that was located in the hips and sacrum was scar healed in a period of less than one month. Product was found effective in lesions of all grades.

### SUMMARY OF THE INVENTION

An illustrative embodiment of the present invention is a composition for topical use to treat wounds which is comprised of dry extract from the bark of the species Stryphnodendron plant; a conservative; a viscosity agent; a pH corrector; and a solvent. In the preferred embodiment, the dry extract is further comprised of between about 1% and about 6% total phenols by volume. These total phenols are preferably comprised of between about 80% and about 90% tannins.

Preferably the phenolic compounds are extracted from the barks of plants from the species Stryphnodendron. It is preferable that the subspecies that supply the phenolic compounds are from the barks of Stryphnodendron polyphyllum or Stryphnodendron adstringens. Additionally it is preferable that the dry extract is obtained through extraction with water, solvents that can be mixed with water, or mixtures of solvents.

Additionally, it is preferred that the conservative agent used is nipagin; the viscosity agent used is glycerin; and the pH corrector used is monosodium phosphate.

In another embodiment, a process for making the composition for topical use to treat wounds is disclosed. Preferably the process includes the following steps: weighing the materials of the composition; adding the materials of the composition together under agitation in specific steps; heating the mixture; cooling the mixture; and adding sufficient solvent to compensate for loss due to evaporation.

### BRIEF DESCRIPTION OF THE DRAWINGS

In order to complement the present description in a way to obtain a better understanding of the characteristics of the present invention and in accordance with a preferable practical accomplishment of the same, a set of drawings accompanies the attached description wherein, by an exemplified way, although not limiting, the following was disclosed:

FIG. 1 is a graph showing the distribution of patients by range of age that were treated with the innovated product in the form of a spray.

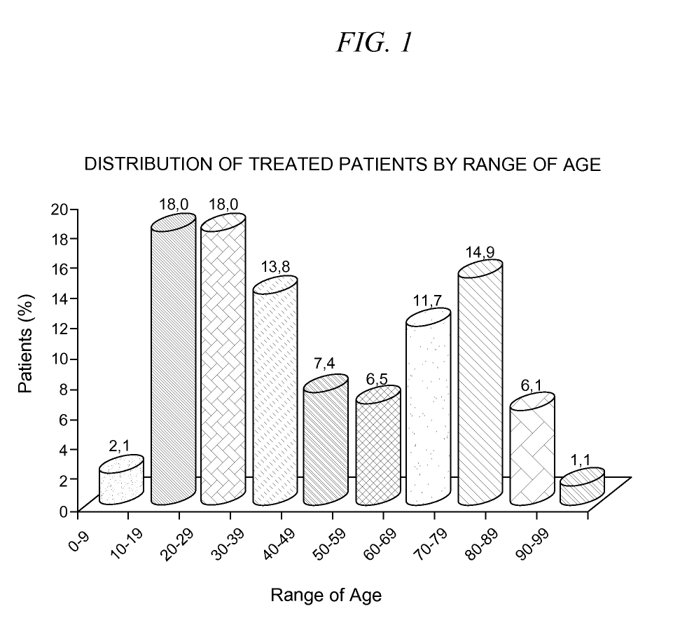

FIG. 2 is a graph showing variation in the number of lesions (pressure ulcers) by treated patients.

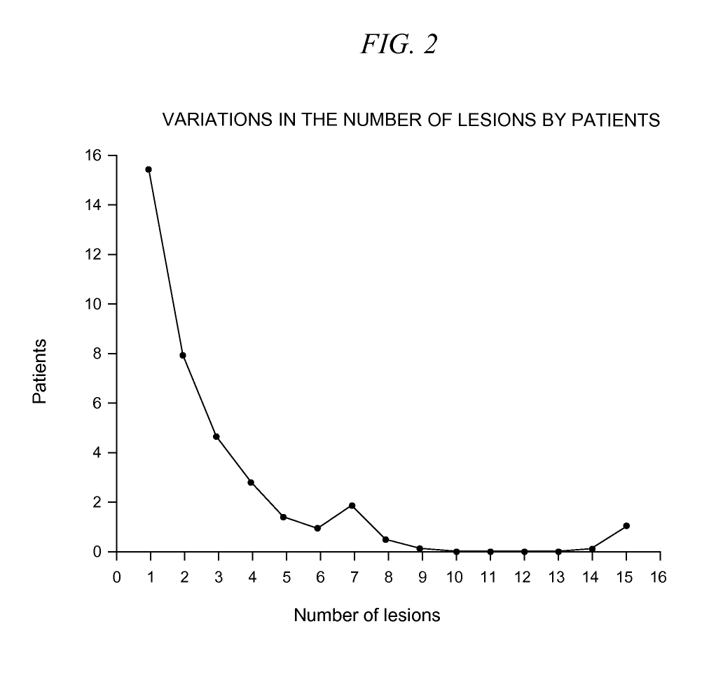

FIG. 3 is a graph showing an evolution in scar healing according to the grade of lesion.

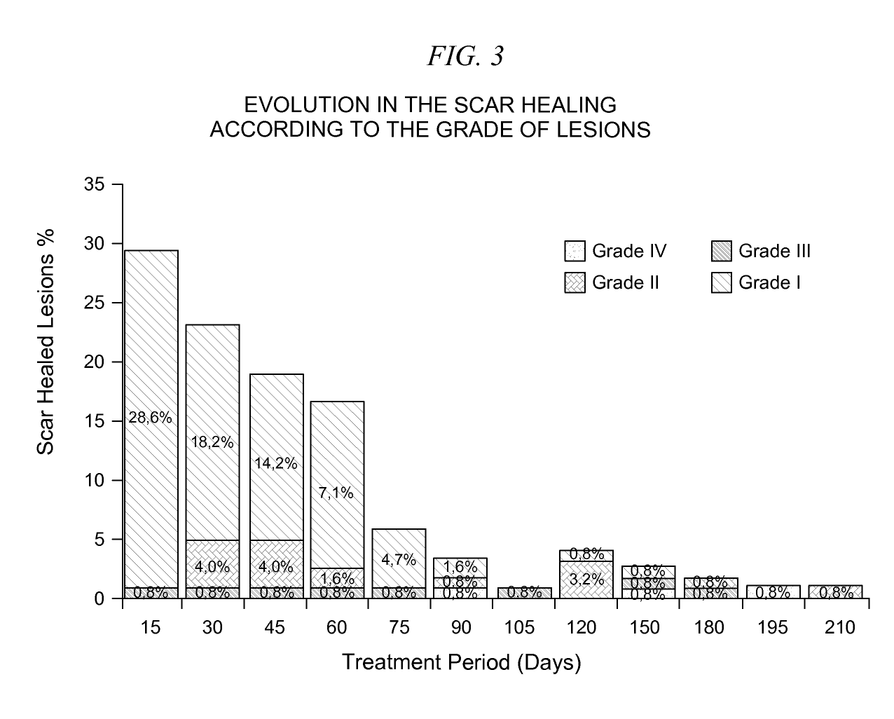

FIG. 4 is a graph showing the variation in the period of treatment by the site of incidence of lesion.

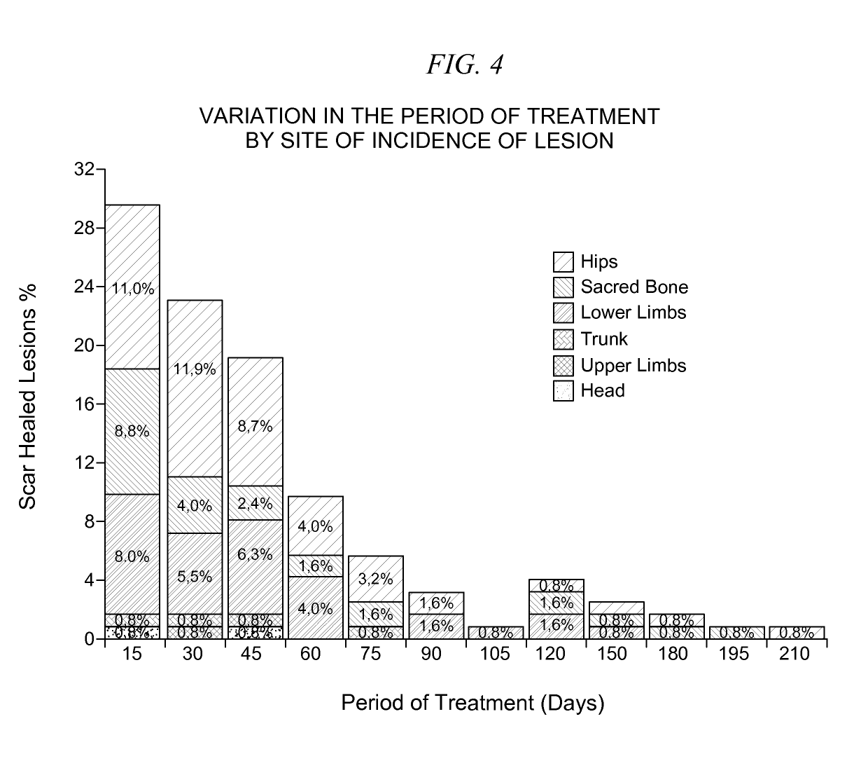

FIG. 5 is a graph showing the distribution of treated lesions by grade.

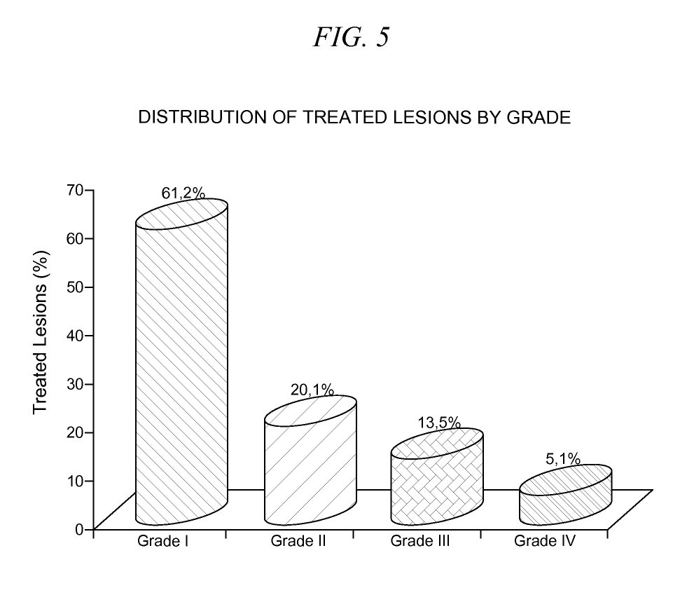

FIG. 6 is a graph showing the efficiency in the scar healing of treated lesions.

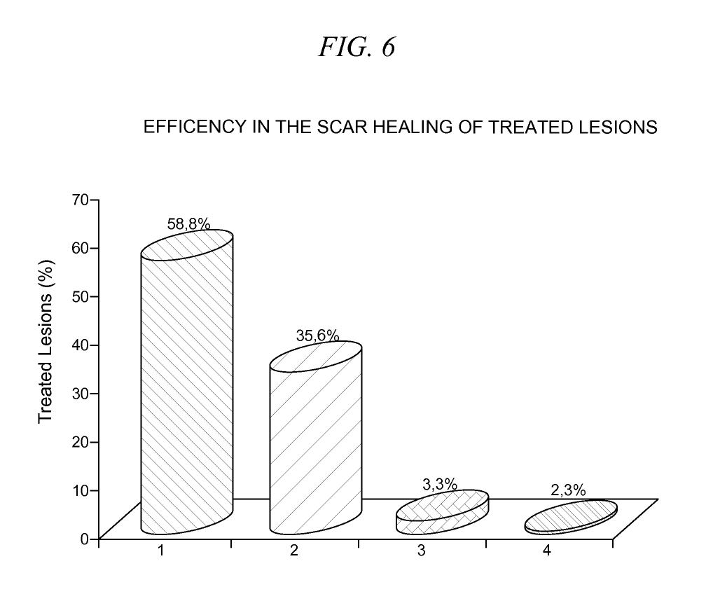

FIG. 7 is a graph showing the distribution of treated lesions by grade.

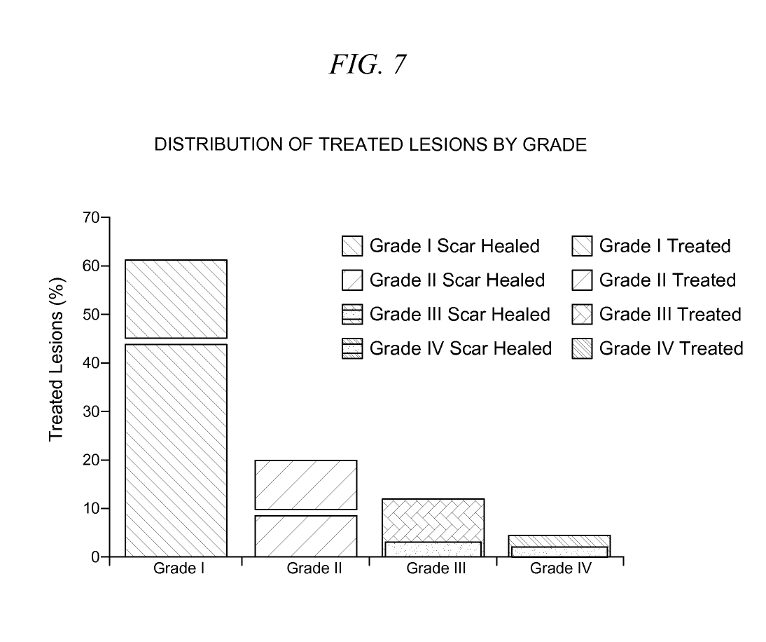

FIG. 8 is a graph showing treatment efficiency of varicose ulcers within a period that ranges from 30 to 210 days as well as the efficiency in connection with the grades of classification.

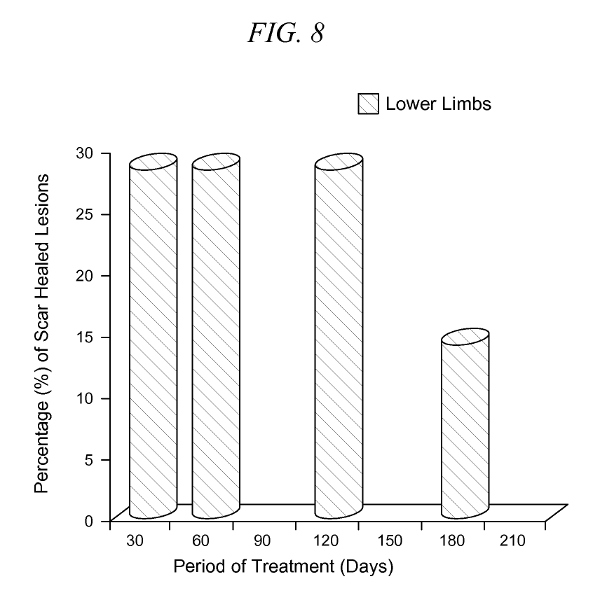

### DETAILED DESCRIPTION OF THE INVENTION

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

The present invention refers to a Phytotherapeutic Pharmaceutical Form with Scar Healing and Microbial ‘Properties and Other Activities of Medical and Veterinary Interest for the Treatment of Ulcers (that either obstruct or decrease the blood irrigation) and Infections; Its Process of Preparation and its Applications. The present invention discloses a phytotherapeutical formulation applied in the treatment, acceleration of scar healing of lesions and revitalization of the damaged tissue in humans and animals through the topical administration of bioactives derived from plants known as Barbatiman of the species Stryphnodendron and subspecies polyphyllum and adstringens. The formulation stimulates the normal synthesis of collagen with an effective action of tissue recovery and regeneration. The formulation also stimulates antimicrobial action on multiresistant field species colonizers of obstructing lesions (pressure and/or varicose); still lesions provoked by surgery; ulcerations due to burns; and infections located in the lesions.

The phytotherapeutical intermediary preparations of Barbatiman were carried out following determined steps and described next obtaining the following physical-chemical characteristics:

Preparation of Stryphnodendron's Dyes:

Dyes are prepared with the ground barks of the plant, soaked for 15 days in a mixture of cereals alcohol in water (2:1) with determined volume. After soaking, mixtures are filtered and the obtained volume is completed for the sufficient quantity.

Preparation of Concentrated Stryphnodendron's Dyes:

Dyes are concentrated in a rotoelevador under vacuum at a maximum temperature of 600 Celsius, being designed by the solutions' concentrations to levels of 50%, 25% and 10%, respectively as solutions A, B and C. The final aspect of dyes which is comprised of a liquid of dark brown red or strawberry red color. The dyes when diluted have no peculiar odor and its taste is of binding sensation. Ph is 4.0-4.5 and the contents of total phenols is of 1.70-1.75 g/100 mL.

Dry Extract of Stryphnodendron:

The dry extracts of Stryphnodendron are obtained from the drought of dyes previously separated and placed in open recipients which provide a better evaporation of solvent. They are left for 5 days in a proofer with air forced circulation at a temperature of 50 degrees. Final aspect comprises a dark brown red color powder that is a strawberry red color when dissolved in an aqueous vehicle. It has no peculiar odor and its taste is binding sensation. Total phenols content: 60.0-65.0 g/100 g.

The formulation developed for the preparation of the phytotherapeutic product contains the dry extract from the Stryphnodendron's bark dye and the assistant substances Nipagin (conservative), glycerin (viscosity agent) monosodium phosphate (pH correction), the following formulation being defined as:

Stryphnodendron's dry extract . . . from 2% to 5% of total phenols and 1.8% to 40% of tannins;

| | |
|---|---|
| Glycerin | 1.0%; |
| Nipagin | 0.2% |
| Phosphate Tissue | q.s. pH 5.5-6.0; and |
| Purified Water | 100.0 mL |

In an ideal concentration the formulation determines that the Stryphnodendron's quantity of dry extract should be of 4.4 to 5.0 g/100 mL to generate a concentration of 60.0 to 65.0 g/100 g of total phenols, that is, 3% of total phenols by volume in the formulated products.

The studied chemical portions were the proanthocyanidins, prorobinetinidins, flavan-3-ols, and prodelfinidins. The anti-inflammatory activity of Stryphnodendron is attributed to the presence of proanthocyanidins.

The topical composition may use vehicles like gels, sprays, creams, ointment and any other that will maintain the wound with certain moisture thus favoring the activity of the innovated phytotherapeutical medicine.

The manufacturing process of the above-referenced formulation will undergo the following steps:
1—Weighing the determined quantities of Stryphnodendron's dry extract, glycerin, nipagin and measuring the quantity of purified water;
2—Placing the Stryphnodendron's dry extract and the glycerin in a tank that holds an adequate mixer;
3—Adding part of distilled water and begin stirring;
4—Adding the nipagin in a part of tepid water;
5—Adding the dissolved nipagin to the main drum of mixture under agitation;
6—Homogenizing for sufficient time until complete dissolution;
7—Adding the remaining volume of water and heating the mixture up to evolution for a 10 minutes then cooling the mixture;
8—Adding the monosodium phosphate under agitation to set up pH around 5.5-6.0;
9 Adding distilled water in the volume of the produced amount in order to compensate for losses due to evaporation;
10—Beginning the filtration with adequate elements and providing samples for analysis at the quality control; and
11—After release, starting up the accommodation of product in the corresponding packages.

The phytotherapeutical formulation for scar healing of topical use now innovated might be used, in an alternate way, in association with physiotherapeutical apparatuses such as ultrasound (US) and low intensity lasers functioning as an accelerator for treatment in the tissue recovery.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

While there has been described an illustrated specific embodiments of a composition for topical use containing an extract of Stryphnodendron its preparation as well as its application, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of treating cutaneous wounds comprising:
contacting the wound with a composition comprised of Stryphnodendron extract wherein the composition is further comprised of about 1% to about 6% total phenols.

2. The method of claim 1 wherein the composition contains about 2% to about 5% total phenols.

3. The method of claim 1 wherein the composition contains about 2.5% to about 3.5% total phenols.

4. The method of claim 1 wherein the total phenols contain about 80% to about 90% tannins.

5. The method of claim 1 wherein the Stryphnodendron extract is obtained from the barks of the Stryphnodendron plant species.

6. The method of claim 5 wherein the Stryphnodendron plant species is selected from the group consisting of Stryphnodendron polyphyllum and Stryphnodendron adsfringens.

7. The method of claim 1 wherein the composition stimulates collagen synthesis.

8. The method of claim 1 wherein the extract is obtained by extraction with water, solvents that can be mixed with water, or mixtures of solvents.

9. A method of treating cutaneous wounds comprising contacting the wound with a composition comprised of Stryphnodendron bark extract wherein the composition contains between about 1% and about 6% total phenols.

10. The method of claim 9 wherein the composition contains about 2% to about 5% total phenols.

11. The method of claim 9 wherein the composition contains about 2.5% to about 3.5% total phenols.

12. The method of claim 9 wherein the total phenols contain about 80% to about 90% tannins.

13. The method of claim 9 wherein the Stryphnodendron plant species is selected from the group consisting of Stryphnodendron polyphyllum and Stryphnodendron adsfringens.

14. The method of claim 9 wherein the composition stimulates collagen synthesis.

15. The method of claim 9 wherein the extract is obtained by extraction with water, solvents that can be mixed with water, or mixtures of solvents.

16. A method of treating cutaneous wounds comprising stimulating collagen synthesis further comprising contacting the wound with a composition comprised of Stryphnodendron bark extract wherein the composition contains between about 1% and about 6% total phenols and between about 80% and about 90% tannins.

17. The method of claim 16 wherein the composition contains about 2% to about 5% total phenols.

18. The method of claim 16 wherein the composition contains about 2.5% to about 3.5% total phenols.

19. The method of claim 16 wherein the Stryphnodendron plant species is selected from the group consisting of Stryphnodendron polyphyllum and Stryphnodendron adsfringens.

20. The method of claim 16 wherein the extract is obtained by extraction with water, solvents that can be mixed with water, or mixtures of solvents.
